(19) United States
(12) Patent Application Publication
(10) Pub. No.: US 2022/0215909 A1
(43) Pub. Date: Jul. 7, 2022

(54) Delta Touchless Range of Motion Analysis

(71) Applicant: ELLIOT RAJ, TAMPA, FL (US)

(72) Inventor: ELLIOT RAJ, TAMPA, FL (US)

(21) Appl. No.: 17/143,580

(22) Filed: Jan. 7, 2021

(51) Int. Cl. G16H 10/60; G16H 10/20; G06Q 20/12; G16H 20/30; G16H 15/00; H04L 29/06; A61B 5/11; A61B 5/00

(52) U.S. Cl. CPC G16H 10/60; G16H 10/20; G06Q 20/127; G16H 20/30; G16H 15/00; H04L 63/0428; G16H 50/70; A61B 5/0013; A61B 5/0077; A61B 5/1128; A61B 5/0022; A61B 5/4884; A61B 5/1121

(57) ABSTRACT

A system for recording and distributing patient range of motion related to musculoskeletal therapy data is disclosed. The invention is comprised of a mobile software application (software app) leveraging a mobile device's onboard video camera to record ranges of motion for various body parts during physical rehabilitation (similar to that of a goniometer). The system requires no external components and therapist (herein referred to as the ‘user’) simply points and shoots at the patient, thus it is a touchless process and require no additional equipment for the patient. The data is collected, compared to well documented normal values for age and gender of patients, reports are created and then encrypted and sent to a secure cloud network where it is archived and then made available other stakeholders such as doctors and insurance companies to track said patient's progress.

### CROSS-REFERENCE TO RELATED APPLICATIONS

This application claims the benefit of U.S. Provisional Patent Application No. 63/205,214, filed in 2020.

### FIELD OF THE INVENTION

The present invention generally relates to physical therapy procedures. More specifically, it relates to a touch-less system for recording patient range of motion, using well-known range of motion movements and compares those movements to the well-known and documented averages for humans of certain ages and/or gender.

### BACKGROUND

In the field of physical therapy a goniometer measures range of motion of limbs and joints of the body. These measurements help accurately track progress in a rehabilitation program. When a patient has a decreased range of motion, a therapist assesses the joint before performing an intervention, and continues to use the tool to monitor progress. The therapist can take these ranges of motion measurements at any joint on the human body. For example, when measuring the knee joint, the therapist places the axis (point of rotation) on the lateral epicondyle of the femur, and lines up the stationary arm with the greater trochanter of the femur. Finally, the therapist lines up the moveable arm of the goniometer with the lateral malleolus of the fibula, and records a measurement using the degree scale on the circular portion of the tool. Human error sometimes results in reading accuracy with goniometers. Issues with the intra-measure (between measures) and inter-tester (between clinicians) and some studies suggest that these errors can be anywhere between 5 and 10 degrees. The universal standard goniometer is a plastic or metal tool with 1 degree increments.

More recently, smartphone application developers have created mobile applications that provide the functions of a goniometer. These applications (such as Knee Goniometer and Goniometer Pro) use the accelerometers in phones to calculate joint angles. Recent research supports these applications and their devices as reliable and valid tools with as much accuracy as a universal goniometer. These devices also must be properly-rested directly on the patient and as a patient moves and bends. During these activities, the accelerometers have to choose which part of the “bend” they will rest on, this selection introduces variances and error similar to those found while using goniometers and intra-measures and inter-testers. United States Patent No. US20090005709A1 disclosed a range of motion measurement device using accelerometers. U.S. Pat. No. 5,758,658A disclosed a compact human range of motion measurement system but it does not operate with a mobile device. United States Patent No. US2020335222A1 disclosed a movement feedback device for orthopedic patients using a camera of a user device, of a patient in motion; however, it does not include a distribution method, nor does it measure well known range of motion movements or compare those movements to the well-known and document averages for humans of certain ages and/or gender, and requires the wearing of a wrist device.

Canadian Patent No. CA2846281C disclosed an apparatus and method for analyzing a golf swing but is not related to the medical field. United States Patent No. US20180177450A1 and US20050105772A1 disclosed systems for delivering feedback based on optical motion capture but do they do not include a cloud network, nor is it specifically aimed at providing specific well known and documented range of motions. There will certainly be thousands of applications for measuring human motion with camera, whether face recognition, sports and other performance evaluations, and many, many other applications. This patent request is specifically for measuring range of motion for musculoskeletal therapy, and the tracking and reporting of patients of these well documented range of motion for insurance, doctors and patients.

### SUMMARY OF THE INVENTION

The device herein disclosed and described provides a solution to the shortcomings in the prior art through the disclosure of digital goniometer. An object of the invention is to allow therapists to determine ranges of motion quickly and easily without touching a patient. The system does not rely on external parts or accelerometers to collect range of motion data. The user simply uses the video camera on their mobile device to record their patient's movements. Another object is to automatically document the specific ranges of motion detected and to automatically create reports automatically.

Another object of the invention is to eliminate paper documentation. The range of motion recordings are automatically sent to a cloud network and organized into patient files and eliminates any therapist note taking for this process.

Another object of the invention is to provide a means to distribute range of motion measurements to stakeholders. For example, once a therapist records these ranges they are forwarded to the cloud network. The network then disseminates the information to doctors and insurance companies so they can track patient progress in real time and is compatible with billing and reporting software.

It is briefly noted that upon a reading this disclosure, those skilled in the art will recognize various means for carrying out these intended features of the invention. As such it is to be understood that other methods, applications and systems adapted to the task may be configured to carry out these features and are therefore considered to be within the scope and intent of the present invention, and are anticipated. With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods and systems for carrying out the several purposes of the present disclosed device. It is important, therefore, that the claims be regarded as including such equivalent construction and methodology insofar as they do not depart from the spirit and scope of the present invention. As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements. The objects features, and advantages of the present invention, as well as the advantages thereof over existing prior art, which will become apparent from the description to follow, are accomplished by the improvements described in this specification and hereinafter described in the following detailed description which fully discloses the invention, but should not be considered as placing limitations thereon.

### BRIEF DESCRIPTION OF THE FIGURES

The accompanying drawings, which are incorporated herein and form a part of the specification, illustrate some, but not the only or exclusive, examples of embodiments and/or features.

FIG. 1 shows a perspective view of the invention in use.

FIG. 2 shows stakeholders interacting with the invention software.

FIG. 3 shows the inventive method of the invention.

Other aspects of the present invention shall be more readily understood when considered in conjunction with the accompanying drawings, and the following detailed description, neither of which should be considered limiting.

### DETAILED DESCRIPTION OF FIGURES

In this description, the directional prepositions of up, upwardly, down, downwardly, front, back, top, upper, bottom, lower, left, right and other such terms refer to the device as it is oriented and appears in the drawings and are used for convenience only; they are not intended to be limiting or to imply that the device has to be used or positioned in any particular orientation. Conventional components of the invention are elements that are well-known in the prior art and will not be discussed in detail for this disclosure.

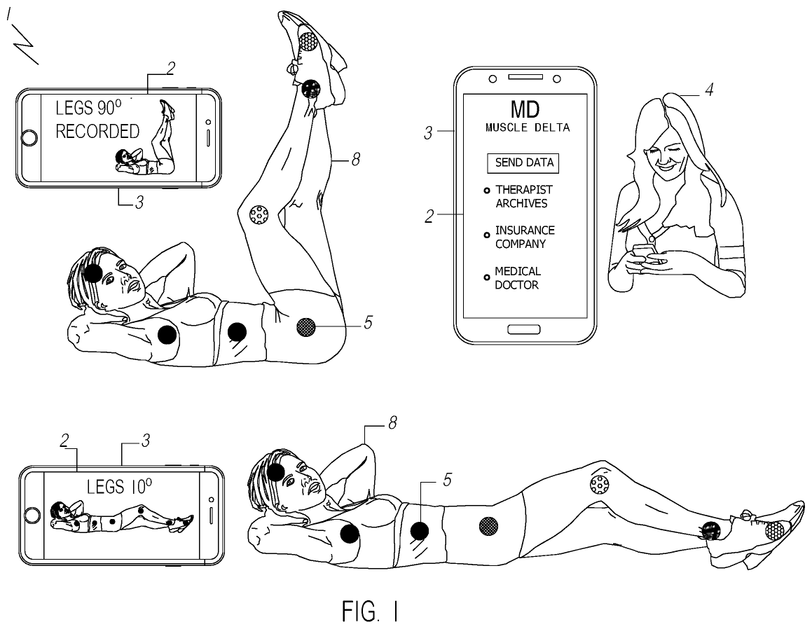

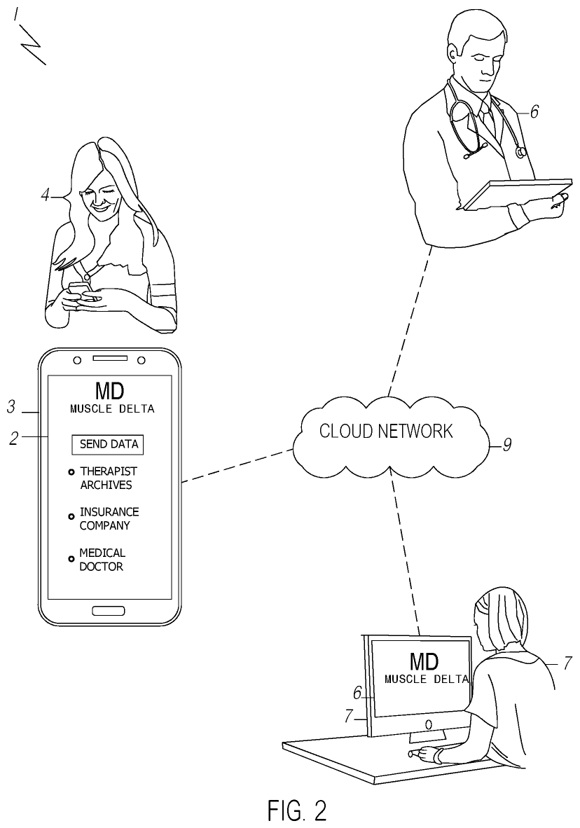

FIGS. 1 and 2 showing various views of a preferred embodiment of the software app 2 being used on smart phone 3 wherein a user (such as a licensed physical therapist etc.) 4 using an existing onboard camera on said smart phone to capture digital images of patient 8. Digital images then being analyzed by an algorithm that identifies joints on a patient's body automatically and superimposes pivot points 5 based on a master list on said images and interpolates angles of range of motion as selected by said physical therapist 4. FIG. 1 showing the user sending range of motion angle information to stakeholders that include but are not limited to other physical therapists, insurance companies and medical health care professionals and the like (doctors etc.).

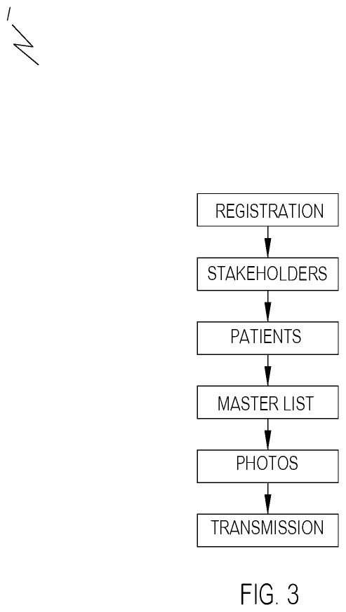

FIG. 2 showing the cloud network 9 receiving information from physical therapist 4 and sending interpolated range of motion angles and images to health care professional 6 and insurance company 7. FIG. 3 showing the steps of the inventive method that include but are not limited to: the user registering their personal demographics, a user name and password, subscription type (monthly annual etc.), and making an online subscription payment (via paypal, venmo etc.); the user then inputting stakeholder information such as doctor contact information, insurance company contact information etc. allowing data from the software app to be transferred when completed; patient data is setup in the software app such as ascribing a patient identification code, recording demographics, gender, height, weight, ailments, medical history, treatment notes etc.; the user selecting a range of motion to measure from a master list (said master list compiled from a plurality of sources such as but not limited to (American Academy of Orthopedic Surgeons, Mayo Clinic, CDC, etc.); the user then taking photos of a patient after showing them a video of the proper body positioning based on said motion measurement from the master list (said photos being taken on the user's mobile device using an onboard camera); the software app identifying pivot points super imposed on patients' bodies in the photos and interpolating an angle from said points and allowing users to add notes and transmit said patient information, recorded range of motion data, patient demographics and user notes to stakeholders.

As used herein, the term engine refers to software, firmware, hardware, or other component that can be used to effectuate a purpose. The engine will typically include software instructions that are stored in non-volatile memory (also referred to as secondary memory). When the software instructions are executed, at least a subset of the software instructions can be loaded into memory (also referred to as primary memory) by a processor. The processor then executes the software instructions in memory. The processor may be a shared processor, a dedicated processor, or a combination of shared or dedicated processors. A typical program will include calls to hardware components (such as I/O devices), which typically requires the execution of drivers. The drivers may or may not be considered part of the engine, but the distinction is not critical.

As used herein a mobile device includes, but is not limited to, a cell phone, such as Apple's iPhone®, other portable electronic devices, such as Apple's iPod Touches®, Apple's iPads®, and mobile devices based on Google's Android® operating system, and any other portable electronic device that includes software, firmware, hardware, or a combination thereof that is capable of at least receiving the signal, decoding if needed, exchanging information with a transaction server to verify the buyer and/or seller's account information, conducting the transaction, and generating a receipt. Typical components of mobile device may include but are not limited to persistent memories like flash ROM, random access memory like SRAM, a camera, a battery, LCD driver, a display, a cellular antenna, a speaker, a BLUETOOTH® circuit, and WIFI circuitry, where the persistent memory may contain programs, applications, and/or an operating system for the mobile device.

As used herein, the terms “social network” and “SNET” comprise a grouping or social structure of devices and/or individuals, as well as connections, links and interdependencies between such devices and/or individuals. Members or actors (including devices) within or affiliated with a SNET may be referred to herein as “nodes”, “social devices”, “SNET members”, “SNET devices”, “user devices” and/or “modules”. In addition, the terms “SNET circle”, “SNET group” and “SNET sub-circle” generally denote a social network that comprises social devices and, as contextually appropriate, human SNET members and personal area networks (“PANs”).

As used herein, the term “computer” is a general purpose device that can be programmed to carry out a finite set of arithmetic or logical operations. Since a sequence of operations can be readily changed, the computer can solve more than one kind of problem. A computer can include of at least one processing element, typically a central processing unit (CPU) and some form of memory. The processing element carries out arithmetic and logic operations, and a sequencing and control unit that can change the order of operations based on stored information. Peripheral devices allow information to be retrieved from an external source, and the result of operations saved and retrieved.

As used herein, the term “Internet” is a global system of interconnected computer networks that use the standard Internet protocol suite (TCP/IP) to serve billions of users worldwide. It is a network of networks that consists of millions of private, public, academic, business, and government networks, of local to global scope, that are linked by a broad array of electronic, wireless and optical networking technologies. The Internet carries an extensive range of information resources and services, such as the inter-linked hypertext documents of the World Wide Web (WWW) and the infrastructure to support email. The communications infrastructure of the Internet consists of its hardware components and a system of software layers that control various aspects of the architecture.

As used herein, the term “extranet” is a computer network that allows controlled access from the outside. An extranet can be an extension of an organization's intranet that is extended to users outside the organization that can be partners, vendors, and suppliers, in isolation from all other Internet users. An extranet can be an intranet mapped onto the public Internet or some other transmission system not accessible to the general public, but managed by more than one company's administrator(s). Examples of extranet-style networks include but are not limited to: LANs or WANs belonging to multiple organizations and interconnected and accessed using remote dial-up LANs or WANs belonging to multiple organizations and interconnected and accessed using dedicated lines. The Virtual private network (VPN) is comprised of LANs or WANs belonging to multiple organizations, and that extends usage to remote users using special “tunneling” software that creates a secure, usually encrypted network connection over public lines, sometimes via an ISP. As used herein, the term “Intranet” is a network that is owned by a single organization that controls its security policies and network management. Examples of intranets include but are not limited to: A Wide-area network (WAN) that is comprised of a LAN that extends usage to remote employees with dial-up access. A WAN that is comprised of interconnected LANs using dedicated communication lines. A Virtual private network (VPN) that is comprised of a LAN or WAN that extends usage to remote employees or networks using special “tunneling” software that creates a secure, usually encrypted connection over public lines, sometimes via an Internet Service Provider (ISP).

For purposes of the present invention, the Internet, extranets and intranets collectively are referred to as (“Network Systems”). In the following description, for purposes of explanation, numerous specific details are set forth in order to provide a thorough understanding of the present invention. However, it will be apparent that the invention may be practiced without these specific details. In other instances, well-known structures and devices are shown in block diagram form in order to facilitate describing the present invention. As used in this application, the terms “component”, “handler”, “model”, “system” and the like are used to refer to computer-related entities, hardware, a combination of hardware and software, software, or software at run time. For example, a component may be, but is not limited to being, a process running on a processor, a processor, an object, an executable, a thread of execution, a program, and a computer. By way of illustration, both an application running on a server and a server can be a component. One or more components can reside within a processor and thread of execution and a component can be localized on one computer and distributed between two or more components. In addition, these components can execute from various computer readable media having various data structures stored thereon. Components may, for example, interact with other systems across a network such as the Internet via one or more data packets (data from one component, and signals that interact with components of other systems in a local or distributed system). Communication via local and remote processes in accordance with signals with data from components). The computer component may be stored, for example, on a computer readable medium, which may be an application specific integrated circuit (ASIC), compact disc (CD), digital video disk (DVD), ROM (ROM) according to the present invention. Read Only Memory, floppy disk, hard disk, electrically erasable programmable read only memory (EEPROM), and memory sticks.

In view of the disclosure provided herein, a mobile application is created by techniques known to those of skill in the art using hardware, languages, and development environments known to the art. Those of skill in the art will recognize that mobile applications are written in several languages include, byway of non-limiting examples, C, C++, C#, Objective-C, Java, Javascript, Pascal, Object Pascal, Python, Ruby, VB.NET, WML, and XHTML/HTML with or without CSS, or combinations thereof. The software also compatible with a plurality of operating systems such as, but not limited to: Windows, Apple, and Android, and are compatible with a multitude of hardware platforms such as, but not limited to: personal desktops, laptops, tablets, smartphones and the like. Suitable mobile application development environments are available from several sources. Commercially available development environments include, by way of non-limiting examples, AirplaySDK, alcheMo, Appcelerator, Celsius, Bedrock, Flash Lite, .NET, Compact Framework, Rhomobile, and Worklight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android, SDK, BlackBerry, SDK, BREW SDK, Palm OS SDK, Symbian SDK, webOS SDK, and Windows Mobile SDK. Those of skill in the art will recognize that several commercial forums are available for distribution of mobile applications including, by way of non-limiting examples, Apple, App Store, Google Play, Chrome Web Store, BlackBerry App World, App Store for Palm devices, App Catalog for webOS, Windows Marketplace for Mobile, Ovi Store for Nokia devices, Samsung Apps, and Nintendo DSi Shop etc.

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java, Lisp, Python, Visual Basic, and VB .NET, or combinations thereof. Compilation is often performed, at least in part, to create an executable program. In some embodiments, a computer program includes one or more executable complied applications.

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe Flash Player, Microsoft Silverlight, and Apple QuickTime.

In view of the disclosure provided herein, those of skill in the art will recognize that several plug-in frameworks are available that enable development of plug-ins in various programming languages, including, by way of non-limiting examples, C++, Delphi, Java, PHP, Python, and VB .NET, or combinations thereof. Web browsers (also called Internet browsers) are software applications, designed for use with network-connected digital processing devices, for retrieving, presenting, and traversing information resources on the World Wide Web. Suitable web browsers include, by way of non-limiting examples, Microsoft Internet Explorer, Mozilla Firefox, Google Chrome, Apple Safari, Opera Software Opera, and KDE Konqueror. In some embodiments, the web browser is a mobile web browser. Mobile web browsers (also called micro-browsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google Android browser, RIM BlackBerry Browser, Apple Safari, Palm Blazer, Palm WebOS Browser, Mozilla Firefox for mobile, Microsoft Internet Explorer Mobile, Amazon Kindle Basic Web, Nokia Browser, Opera Software Opera Mobile, and Sony PSP browser. Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

What is claimed is:

1. A method of measuring range of motion that includes the following inventive steps:
a) providing a computer device having a memory and a camera and including an input means for inputting information, and a display, and loading said computer device with software app for operating said method;
b) providing a secure cloud network connecting to said computer device;
c) providing a software app accessible to said computer device via said secure cloud network whereby said software app facilitates displaying images on said computer device display including:
i) a main page with links to subsequent pages, clicking on said links facilitating access to said subsequent pages;
ii) said subsequent pages including:
A) a first page in which the user registering a user name and password and inputs their information, demographics and subscription information;
B) a second page in which the user entering stakeholders' information and demographics;
C) a third page in which the user entering patient information and demographics;
D) a fourth page in which the user searching a master list of motions to measure;
E) a fifth page in which the user taking photos of the patient and obtaining range of motion angle information; and
F) a sixth page in which the user transmitting photos and range of motion angle information to stakeholders.

2. The method of measuring range of motion of claim 1 wherein the first page in which the user registering a user name and password and inputs their information, demographics and subscription information into the software app includes the step of paying for said subscription by means of an online payment method said user name and password, information, demographics and subscription information includes the step of inputting said profiles into said computer device memory and said secure cloud network memory.

3. The method of measuring range of motion of claim 1 wherein the second page in which the user entering stakeholders' information and demographics includes the step of inputting information and demographics of stakeholders that include but are not limited to other therapists, doctors and insurance companies into said computer device memory and said secure cloud network memory.

4. The method of measuring range of motion of claim 1 wherein the third page in which the user entering patient information and demographics includes the step of inputting said profiles that includes information such as but not limited to age, gender, height, weight, medical history and ailments into said computer device memory and said secure cloud network memory.

5. The method of measuring range of motion of claim 1 wherein the fifth page in which the user taking photos of the patient and obtaining range of motion angle information includes the step of the software app providing the user with instructions on how to capture range of motion photos.

6. The method of measuring range of motion of claim 1 wherein the fifth page in which the user taking photos of the patient and obtaining range of motion angle information includes the step of showing the patient a video of the motion selected from the master list and having the patient replicate the motion from said master list.

7. The method of measuring range of motion of claim 1 wherein the fifth page in which the user taking photos of the patient and obtaining range of motion angle information includes the step of an algorithm within said software app identifying pivot points super imposed on patients' bodies and interpolating an angle from said points.

8. The method of measuring range of motion of claim 1 wherein the fifth page in which the user taking photos of the patient and obtaining range of motion angle information includes the step of adding notes and inputting said taken photos and notes into said computer device memory and said secure cloud network memory.

9. The method of measuring range of motion of claim 1 wherein the sixth page in which the user transmits photos and range of motion angle information to stakeholders includes the step of encrypting motion angle information and said notes and transmitting into said computer device memory and said secure cloud network memory.
